(19) United States
(12) Patent Application Publication
(10) Pub. No.: US 2026/0083903 A1
(43) Pub. Date: Mar. 26, 2026

(54) INFUSION PUMP WITH AIR BUBBLE DETECTION AND METHOD FOR OPERATING AN INFUSION PUMP FOR AIR BUBBLE DETECTION

(71) Applicant: B. Braun Melsungen AG, Melsungen (DE)

(72) Inventors: Jan Schwarz, Melsungen (DE); Sebastian Moeller, Hessisch Lichtenau (DE)

(21) Appl. No.: 19/333,625

(22) Filed: Sep. 19, 2025

(30) Foreign Application Priority Data
Sep. 23, 2024 (EP) 24201886.9

(51) Int. Cl. A61M 5/142; A61M 5/168

(52) U.S. Cl. CPC A61M 5/14212; A61M 5/16831; A61M 2205/106; A61M 2205/3313; A61M 2205/3375

(57) ABSTRACT

An infusion pump has a sensor arrangement and an infusion line or a holding device with a holding region for an infusion line. An ultrasonic sensor and an imaging device with a camera are arranged on the outer circumference of the holding region of the holding device or on the outer circumference of the infusion line. At least the imaging device is configured to detect gas bubbles and, preferably, to detect particles in an infusion fluid. At least the imaging device is preferably configured to detect a size and to detect a change in the size of the gas bubbles and/or to detect movement of the gas bubbles, and preferably particles. The ultrasonic sensor preferably performs an initialization by detecting the infusion fluid in the infusion line and, if necessary, detecting gas bubbles.

### CROSS-REFERENCE TO RELATED APPLICATION

This application claims priority under 35 U.S.C. § 119 to European Application No. 24201886.9, filed on Sep. 23, 2024, the content of which is incorporated by reference herein in its entirety.

### FIELD

The present disclosure relates to the detection of air bubbles and, preferably, additional particle detection in a medical infusion fluid in an infusion line, preferably in an infusion tube of an infusion pump, preferably a peristaltic pump.

### BACKGROUND

In medicine, liquid medications are administered to patients using infusion pumps and injected into their bloodstream either continuously or intermittently. It is important that no air bubbles (or other gas bubbles) are introduced together with the liquid medication.

The affected infusion pump may be a peristaltic pump or may contain one. A tube segment (pump segment) is inserted into such an infusion pump, in which the infusion fluid is further conveyed, for example by means of external rollers, and into which a further tube segment (sensor segment) is inserted, at which the air bubble detection is carried out.

The applicant's EP 3 296 717 A1 discloses an optical imaging sensor for a dialysis machine tube, wherein the sensor detects foreign substances such as impurities and air bubbles. The state of the art of the applicant also includes air bubble sensors that can detect air bubbles based on the emission of an ultrasonic signal with a frequency. This approach has limited resolution depending on the selected frequency and therefore only detects air bubbles above a certain size.

CN 112107758 B discloses a sensor arrangement for an infusion pump, which is set up as an ultrasonic sensor for different frequencies. Air bubble detection with more than one frequency has the advantage that different degrees of accuracy are possible, meaning that different air bubble sizes can be detected based on the selected frequency.

State-of-the-art ultrasonic sensors for infusion pumps are capable of detecting air bubbles in an infusion tube filled with infusion fluid, but due to the frequency selected, they are limited in terms of the size of air bubbles that can be detected.

### SUMMARY

The purpose of this disclosure is to provide an infusion pump, a method, and a computer-implemented storage medium in which air bubble detection is improved. In particular, the limitation to detectable air bubble sizes should be overcome.

This task is solved with regard to the infusion pump with the combination of features of claim 1, with regard to the method with the combination of features of claim 11, and with regard to a computer-implemented storage medium or a control unit with the combination of features of claim 16.

The infusion pump according to the disclosure has an infusion line or a holding device with a holding region for an infusion line. The infusion line is preferably an infusion tube. An imaging device with a camera and an ultrasonic sensor are arranged on the outer circumference of the holding region and/or the infusion line, wherein at least the imaging device is configured and set up to detect air bubbles or other gas bubbles and preferably (additionally) to detect particles of an infusion fluid. The infusion fluid is contained in the infusion line and can flow through it or remain stationary. The imaging device is capable of detecting when a gas bubble occurs in the infusion fluid by means of image processing. It can also detect whether the gas bubble is moving, whether it is getting larger over time, or whether the air bubble is stationary. In the latter case, beyond gas bubble detection, it can be concluded that the gas bubble has become stuck or that the (entire) infusion fluid is in the infusion line. In the first case, an alarm can be prevented. In the second case, this additional information can also be used by the affected infusion pump as information on (higher-level) pump operation.

The optional ultrasonic sensor can be set up and configured in various ways:
The ultrasonic sensor can be set up and configured to perform only an initialization, during which it detects the presence of the infusion fluid in the infusion line. For this purpose, the ultrasonic sensor is set up and configured to distinguish between air and infusion fluid.
The ultrasonic sensor can be set up and configured to assist in gas bubble detection after initialization or, in special cases (e.g., with a light-absorbing infusion line and an imaging device based on visible light), to perform gas bubble detection on its own.
It is also possible that the imaging device itself is set up and configured for initialization and that the ultrasonic sensor assists in gas bubble detection or takes over this function in special cases. For this purpose, the imaging device and the ultrasonic sensor are set up and configured to detect gas bubbles in the infusion fluid.

Accordingly (to the above case distinction), an electronic control unit of the infusion pump can be set up and configured as follows:
The control unit can be set up and configured to control only initialization via the ultrasonic sensor, wherein the presence of the infusion fluid in the infusion line is detected, and then to control gas bubble detection via the imaging device.
The control unit can be set up and configured to control the initialization via the ultrasonic sensor and then, together with the imaging device, to control the gas bubble detection or, in special cases (e.g., with a light-absorbing infusion line and an imaging device based on visible light), to perform the gas bubble detection using only the ultrasonic sensor.
The control unit can be set up and configured to control initialization via the imaging device and then to control gas bubble detection, e.g., redundantly, using the ultrasonic sensor and the imaging device.

The holding device is configured and set up for temporary holding and fixing of the infusion line, preferably the infusion tube. For this purpose, the holding device has a holding region into which the infusion line can be inserted and fixed, e.g., clamped and/or clamped in place. The infusion line can then be designed as an infusion tube and replaced as a disposable item when the infusion fluid is changed. The holding device may have a cover for securing the object.

If the camera is based on visible light, and particularly in the case of the cover or front flap of a housing of the infusion pump concerned, it is preferable, due to the darkening effect, for a light source, particularly an LED, to be arranged on the outer circumference of the holding region or the infusion line. The light source is preferably arranged on one side of the holding region or the infusion line opposite the camera.

Alternatively, the camera can also be configured and set up to detect infrared rays/infrared radiation. The imaging device can then visualize and detect different gas bubbles based on the thermal radiation.

Of course, it is particularly preferable if the infusion line is translucent. However, restrictions in the light transmission of the infusion line are also possible, which can be compensated (at least partially) with the additional ultrasonic sensor.

The sensor arrangement can be compact and/or require only a common holding device for the infusion line if the ultrasonic sensor is arranged adjacent to the imaging device.

If it is advantageous for the design of the higher-level infusion pump, the ultrasonic sensor can also be arranged along the infusion line at a distance from the imaging device. In the case of the infusion tube, the ultrasonic sensor has its own holding device for the infusion tube.

The ultrasonic sensor can be set up for one frequency or for several or different frequencies. Air bubble detection can be further improved according to a preferred exemplary embodiment, if the ultrasonic sensor is set up for different frequencies.

Another design takes into account that the imaging device is susceptible to contamination and that infusion tubes are available on the market that have UV protection consisting of a light-absorbing or light-impermeable coating. Then the imaging device may reach its limits. It is therefore particularly advantageous if the ultrasonic sensor is also set up and configured to detect gas bubbles and, preferably, particles in the infusion fluid. The ultrasonic sensor can then detect the gas bubbles and, preferably, the particles in the cases mentioned.

Imaging also enables differentiation between the infusion fluids in the infusion tube, e.g., through edge detection and/or pattern detection. By detecting a change, it is possible to detect/estimate the size of an air bubble or a particle, preferably after initialization, in particular by the ultrasonic sensor. Initialization by the ultrasonic sensor has the advantage that image processing has less work to do, and imaging can then work much better, especially with UV-coated infusion tubes. Since imaging has a larger detection range, it can also detect changes over time in order to detect the movement of gas bubbles/particles or growth.

In a preferred further development of the infusion pump, the holding device for the infusion tube is attached to a housing of the infusion pump.

Furthermore, the front flap of the infusion pump or, at least indirectly, the cover of the sensor arrangement can also be attached to the housing. Especially if the camera is set up and configured for visible light, it is preferable to use the above-mentioned light source due to the darkening.

The infusion pump in question is or preferably has a peristaltic pump (tube pump/tube roller pump/tube squeeze pump) that has an infusion tube segment (pump segment) in which the infusion fluid is (further) conveyed by means of external mechanical deformation, for example by means of external rollers, in particular by being pressed through the infusion tube segment. The infusion tube segment can be formed together with the sensor segment, on which the sensor arrangement is arranged, as a single-piece infusion tube, or it can consist of two or more separate tube segments connected by a coupling.

The infusion pump therefore preferably has three components that are arranged directly on the infusion tube, namely means for external mechanical deformation, for example rollers or plungers, the peristaltic pump, the imaging device, and the ultrasonic sensor. These three components can also be arranged spaced apart from each other on different tube segments of an infusion tube. Then each of these components has its own holding device.

In one embodiment, the ultrasonic sensor is specifically configured and set up to distinguish between the infusion fluid and air in the infusion line. The imaging device can then be initialized with the ultrasonic sensor.

The method according to the disclosure serves to operate the sensor arrangement described above and comprises the steps of “initialization”and “gas bubble detection”.

In one exemplary embodiment, the imaging device is initialized with the ultrasonic sensor, in particular activated/made ready for operation.

After initialization, the imaging device and the ultrasonic sensor can redundantly detect air bubbles or other gas bubbles and preferably particles.

Alternatively, in gas bubble detection, the ultrasonic sensor may (only) detect the presence or existence of a gas bubble, in particular an air bubble, and the imaging device may then detect the growth and/or movement of the gas bubble.

If the existence of a gas bubble is detected but no movement of the gas bubble is detected, an alarm or error message can be prevented because the gas bubble has become stuck to the tube.

The computer-implemented storage medium or electronic control unit disclosed in the disclosure is set up, configured, and programmed to perform the aforementioned method steps.

### BRIEF DESCRIPTION OF THE DRAWINGS

FIG. 1 shows an infusion pump according to an exemplary embodiment of the present disclosure with further components in a schematic overview;

FIG. 2 shows a further illustration of the exemplary embodiment of the infusion pump from FIG. 1; and

FIG. 3 shows the method for operating the infusion pump from FIG. 1.

### DETAILED DESCRIPTION

The following describes an exemplary embodiment of the infusion pump and three exemplary embodiments of the method according to the present disclosure based on the accompanying figures.

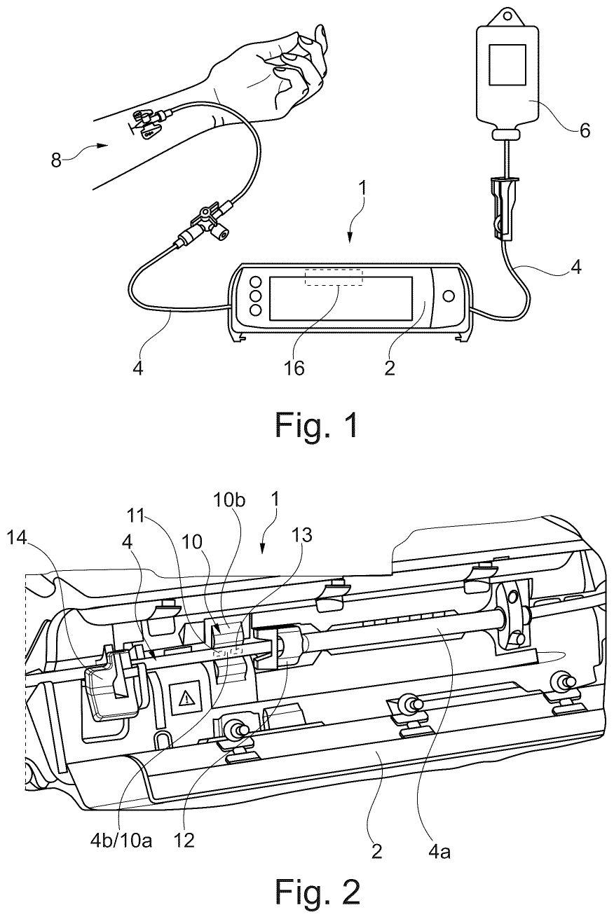

FIG. 1 shows an infusion pump 1 according to an exemplary embodiment of the present disclosure. Its housing has a front flap 2, which partially covers a multi-part infusion tube 4, among other things. The infusion tube 4 has at least two separate tube segments connected by respective couplings. A liquid medication is delivered from a bag 6 to a patient's access point 8 via the infusion tube 4. For this purpose, the infusion pump 1 is designed with a peristaltic pump that engages with the infusion tube 4 by means of external mechanical deformation and compresses or squeezes it. The compressed section of the infusion tube 4 moves (in FIG. 1 from right to left) along the infusion tube 4 toward the patient 8.

FIG. 2 shows another view of the exemplary embodiment of the infusion pump 1 from FIG. 1 with the front flap 2 open. The infusion tube 4 extends behind the flap 2 through the infusion pump 1 or through its housing. The infusion tube 4 has a pump segment 4a in the region where the peristalsis takes effect.

In the exemplary embodiment shown on the patient side (i.e., in FIG. 2 on the left) of pump segment 4a, a sensor segment 4b of infusion tube 4 is fixed, on the outer circumference of which a holding device 10 of a sensor arrangement is arranged. More precisely, the holding device 10 has two jaws 10b between which the sensor segment 4b of the infusion tube 4 is detachably fixed (e.g., clipped in).

A camera and an LED light source are integrated into the holding device 10, forming an imaging device 11. Furthermore, an ultrasonic sensor 13 is integrated into the holding device 10. The imaging device 11 and the ultrasonic sensor 13 together form the sensor arrangement according to the disclosure.

A coupling 12 is provided between the two segments 4a, 4b of the infusion tube 4.

On the patient side of the sensor arrangement with the imaging device 11 and the ultrasonic sensor 13, a clamp 14 (free flow protection clamp, FFPC) is also arranged on the infusion tube 4. This is in two parts, with one part fixed to the inner infusion tube 4 and the other part fixed to the housing of the infusion pump 1.

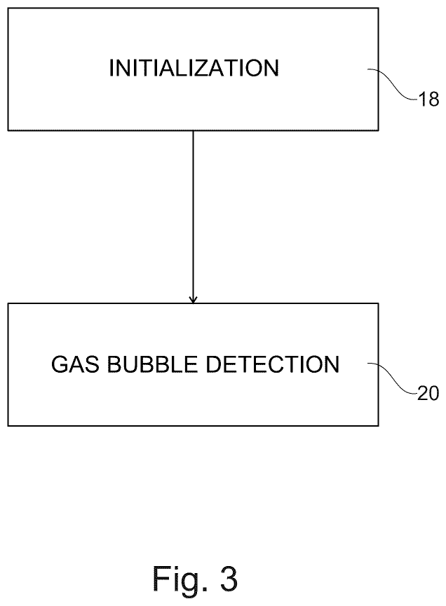

With the infusion pump 1 shown in FIGS. 1 and 2, which is in accordance with the disclosure, various exemplary embodiments of the method for detecting air bubbles and particles can be carried out, which are controlled accordingly by an electronic control unit 16 (shown schematically in FIG. 1). Three different exemplary embodiments of the method are shown schematically together in FIG. 3. They each consist of an initialization 18 and a gas bubble detection 20.

According to a first exemplary embodiment, the control unit 16 controls only the initialization 18 via the ultrasonic sensor 13, wherein the presence of the infusion fluid in the infusion line 4 is detected. The control unit 16 then controls the gas bubble detection 20 via the imaging device 11.

According to another exemplary embodiment, the control unit 16 controls the initialization 18 via the ultrasonic sensor 13. The control unit 16 then controls the gas bubble detection 20 via the ultrasonic sensor 13 together with the imaging device 11. During transition, e.g., in the case of a light-absorbing infusion line 4, gas bubble detection 20 can only be performed by the ultrasonic sensor 13.

According to a further exemplary embodiment, the control unit 16 controls the initialization 18 and then the gas bubble detection 20 of the ultrasonic sensor 13 together with the imaging device 11 via the imaging device 11.

An infusion pump 1 with a sensor arrangement 11, 13 and a method for operating the sensor arrangement 11, 13 are disclosed. The sensor arrangement 11, 13 has an infusion line 4 or a holding device 10 with a holding region 10a for an infusion line 4. An imaging device 11 with a camera is arranged on the outer circumference of the holding region 10a of the holding device 10 or on the outer circumference of the infusion line 4, which is set up and configured for detecting 20 air bubbles or other gas bubbles and preferably for detecting particles in an infusion fluid. The imaging device 11 is set up and configured to detect a size and to detect a change in the size (enlargement/reduction) of the gas bubbles. Furthermore, the imaging device 11 is configured and set up to detect the movement of the gas bubbles and, preferably, the particles. In addition, an ultrasonic sensor 13 is provided, which preferably performs initialization 18 by detecting the infusion fluid in the infusion line 4 and, if necessary, gas bubble detection 20.

The camera can be set up for visible light or for invisible light, e.g., infrared.

### LIST OF REFERENCE DESIGNATIONS

1 infusion pump
2 front flap
4 infusion line/(multi-part) infusion tube
4a pump segment
4b sensor segment
6 bags
8 patient
10 holding device
10a holding region
10b jaw
11 Imaging device
12 coupling
13 ultrasonic sensor
14 clamp
16 electronic control unit
18 initialization
20 gas bubble detection

1. An infusion pump comprising:
an infusion line or a holding device with a holding region for an infusion line;
an ultrasonic sensor; and
an imaging device with a camera,
the ultrasonic sensor and the imaging device being arranged on an outer circumference of the infusion line or the holding region, and
at least the imaging device being configured to detect gas bubbles in an infusion fluid.

2. The infusion pump according to claim 1, wherein at least the imaging device is further configured to detect particles in the infusion fluid.

3. The infusion pump according to claim 1, wherein at least the imaging device is configured to detect a size of the gas bubbles.

4. The infusion pump according to claim 1, wherein at least the imaging device is configured to detect at least one of:
a change in size of the gas bubbles, and
movement of the gas bubbles.

5. The infusion pump according to claim 1, wherein the ultrasonic sensor is configured for initialization, including at detection of at least one of:
the infusion fluid in the infusion line, and
the gas bubbles in the infusion fluid.

6. The infusion pump according to claim 1, wherein the imaging device has a light source arranged on an outer circumference of the holding region or on an outer circumference of the infusion line.

7. The infusion pump according to claim 6, wherein the light source is arranged on a side of the holding region opposite the camera or on a side of the infusion line opposite the camera.

8. The infusion pump according to claim 1, wherein the camera is configured to detect infrared radiation.

9. The infusion pump according to claim 1, wherein the infusion line is light-transmissive or light-absorbing.

10. The infusion pump according to claim 1, further comprising a housing on which a front flap is hinged, the front flap configured to cover the imaging device.

11. The infusion pump according to claim 1, wherein the ultrasonic sensor is configured for one or more frequencies.

12. The infusion pump according to claim 1, wherein:
the infusion pump is or comprises a peristaltic pump,
the infusion pump comprises an infusion tube, and
the infusion line is the infusion tube.

13. The infusion pump according to claim 1, wherein the imaging device is configured for initialization, including detection of the infusion fluid in the infusion line.

14. A method for operating an infusion pump, the method comprising the steps of:
initializing an imaging device by detecting an infusion fluid in an infusion line via an ultrasonic sensor or the imaging device; and
detecting gas bubbles in the infusion fluid via the ultrasonic sensor and/or the imaging device.

15. The method according to claim 14, further comprising the step of detecting a size of the gas bubbles and/or movement of the gas bubbles.

16. The method according to claim 14, further comprising the step of detecting a change in size of the gas bubbles.

17. The method according to claim 14, wherein the step of initializing the imaging device is performed by the ultrasonic sensor and the step of detecting gas bubbles is performed by the imaging device.

18. The method according to claim 17, wherein the step of detecting gas bubbles is further performed by the ultrasonic sensor.

19. The method according to claim 14, further comprising the step of detecting particles in the infusion fluid.

20. A computer-implemented non-transitory storage medium or electronic control unit configured to carry out the method according to claim 14.
